(19) United States
(12) Patent Application Publication
(10) Pub. No.: US 2014/0171632 A1
(43) Pub. Date: Jun. 19, 2014

(54) APPARATUS AND METHOD FOR SEPARATING FIBROUS MATERIAL

(71) Applicant: GWANGJU INSTITUTE OF SCIENCE AND TECHNOLOGY, Gwangju (KR)

(72) Inventor: Yong-Gu Lee, Gwangju (KR)

(73) Assignee: GWANGJU INSTITUTE OF SCIENCE AND TECHNOLOGY, Gwangju (KR)

(21) Appl. No.: 14/104,862

(22) Filed: Dec. 12, 2013

(30) Foreign Application Priority Data
Dec. 13, 2012 (KR) 10-2012-0145006

(51) Int. Cl. C07H 21/04; B03C 5/00

(52) U.S. Cl. CPC C07H 21/04; B03C 5/00
USPC 536/23.1; 204/660; 204/554; 422/255

(57) ABSTRACT

An apparatus for separating fibrous materials includes: an introduction unit through which fibrous materials capable of performing Brownian motion are introduced; a moving path providing section prepared in a state in which at least one field causing movement of the fibrous materials in a predetermined direction is applied thereto, and providing a moving path for the fibrous materials; at least three capture zone-providing segments provided to the moving path providing section so as to at least temporarily capture the fibrous materials; and an collecting unit collecting the fibrous materials having passed through the moving path providing section. The apparatus and method have improved safety and improved purity of separated fibrous materials, enables separation of large amounts of fibrous materials through a simple separation process, and the apparatus and method have other various advantages inferred from the spirit of the invention. Therefore, the apparatus and method may significantly increase yield.

### CROSS-REFERENCE TO RELATED APPLICATION

This application claims priority to Korean Patent Application No. 10-2012-0145006 filed on 13 Dec. 2012, and all the benefits accruing therefrom under 35 U.S.C. §119, the contents of which is incorporated by reference in its entirety.

### BACKGROUND

1. Technical Field

The present invention relates to an apparatus and method for separating fibrous materials. More particularly, the present invention relates to an apparatus and method for separating mixed materials capable of performing Brownian motion according to the kind of material.

2. Description of the Related Art

As a method for separating fibrous materials such as DNA, gel electrophoresis is known in the art. In gel electrophoresis, DNA is stained with ethidium bromide and is then passed through gel to be fractionated. Then, after gel at a stained position is cut away under UV irradiation, gel and dye are removed and, as a result, DNA can be extracted.

Gel electrophoresis has a safety problem due to the use of toxic dyes, can destroy fragment DNA due to chemical treatment, and is time consuming due to the use of several stages. Moreover, although gel electrophoresis can be applied to an examination scale in which an extremely small amount of material to be examined is used, it cannot be used for industrial purposes because the respective stages are separate and cannot be continuously performed.

### BRIEF SUMMARY

The present invention is conceived to solve the above problems, and aspects of the present invention are to provide an apparatus and method for separating fibrous materials, which can be safely applied due to non-toxicity and no cause of pollution, can separate a fibrous material such as DNA in a pure state, can separate a large amount of fibrous materials through simple, rapid and continuous processes.

In accordance with one aspect of the present invention, an apparatus for separating fibrous materials includes: an introduction unit through which fibrous materials capable of performing Brownian motion are introduced; a moving path providing section prepared in a state in which at least one field causing movement of the fibrous materials in a predetermined direction is applied thereto, and providing a moving path for the fibrous materials; at least three capture zone-providing segments provided to the moving path providing section so as to at least temporarily capture the fibrous materials; and a collecting unit collecting the fibrous materials having passed through the moving path providing section.

The at least one field may be at least one of an electric field, a magnetic field, a gravitational field, and a fluid field, and capture zones provided by the at least three capture zone-providing segments may be formed by at least one of a light field due to a nanoplasmon phenomenon, a fluid field, and a DNA binding protein, and a distance between the at least three capture zone-providing segments close to a direction of applying the at least one field may be greater than that far from the applied direction of the at least one field.

In accordance with another aspect of the present invention, a method for separating fibrous materials includes: moving fibrous materials along a field; and at least temporarily capturing the fibrous materials in at least three capture zones separated from each other.

A distance between the at least three capture zones close to an applied direction of the field may be larger than that far from the direction of applying the field.

According to embodiments of the invention, the apparatus and method for separating fibrous materials may have improved safety and improved purity of separated fibrous materials, and enables separation of large amounts of fibrous materials through a simple separation process. In addition, the present invention provides various other advantages that can be inferred from the spirit of the invention.

### BRIEF DESCRIPTION OF THE DRAWINGS

The above and other aspects, features, and advantages of the present invention will become apparent from the detailed description of the following embodiments in conjunction with the accompanying drawings, in which:

FIG. 1 is a schematic diagram of an apparatus for separating fibrous materials according to one embodiment of the present invention;

FIG. 2 is a plan view of a moving path providing section;

FIGS. 3 to 6 show a movement process of fibrous materials in a pair of adjacent capture zones depending on time difference;

FIG. 7 is a diagram showing two types of fibrous materials passing through a moving path providing section;

FIG. 8 is a plan view of a moving path providing section according to another embodiment of the present invention; and

FIG. 9 is a flowchart of a method for separating fibrous materials.

### DETAILED DESCRIPTION

Hereinafter, embodiments of the present invention will be described in detail with reference to the accompanying drawings. It should be understood by those skilled in the art that the present invention is not limited to the following embodiments and may be embodied in different ways, and that various modifications, changes, and alterations can be made without departing from the spirit and scope of the invention.

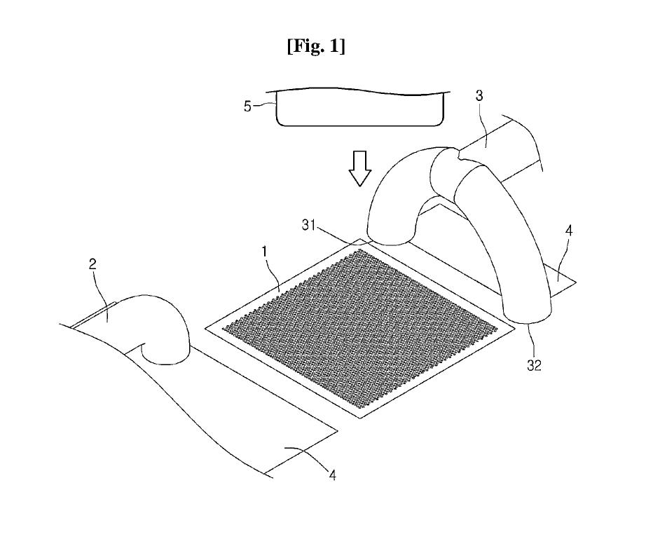

FIG. 1 is a schematic diagram of an apparatus for separating fibrous materials according to one embodiment of the present invention.

Referring to FIG. 1, an apparatus for separating fibrous materials according to one embodiment of the invention includes: an introduction unit 2 through which fibrous materials such as DNA are introduced; a moving path providing section 1 providing a moving path for the fibrous materials introduced through the introduction unit 2; a collecting unit 3 collecting the fibrous materials having passed through the moving path providing section 1, and a field generator 4 at least partially disposed in front of and behind the moving path providing section 1. In addition, the apparatus further includes a laser device 5 irradiating a laser beam toward the moving path providing section 1.

Any device may be used as the introduction unit 2 so long as fibrous materials can be introduced therethrough.

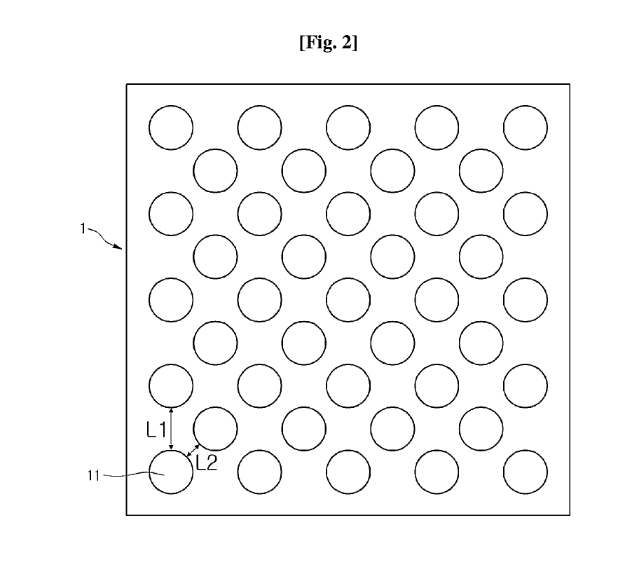

Referring to FIG. 2, the moving path providing section 1 may have a plate shape. For example, the moving path providing section 1 may have a size of 1 mm×1 mm Capture zone-providing segments 11 are arranged in a lattice structure in the moving path providing section 1. The capture zone-providing segments 11 may be prepared as circular holes each having a diameter of 100 nm Instead of the circular shape, the capture zone-providing segments may have other shapes, such as a rectangular shape, triangular shape, and the like. The capture zone-providing segments 11 may function to temporarily capture the fibrous materials. In FIG. 2, the fibrous materials move from a lower side to an upper side. Here, a distance (L1) between the capture zone-providing segments 11 in a direction parallel to a moving direction of the fibrous materials (that is, a vertical direction in FIG. 2) is greater than a distance (L2) between the capture zone-providing segments 11 in a diagonal direction (that is, a diagonal direction in FIG. 2). For example, L1 may be 200 nm and L2 may be 140 nm.

The collecting unit 3 performs operation of collecting the fibrous materials having passed through the moving path providing section 1. The collecting unit 3 may include a first collecting portion 31 and a second collecting portion 32. The first and second collecting portions 31, 32 collect the separated fibrous materials according to the kind of fibrous material, so that each of first and second collecting portions collects the same or different kind of fibrous material. For example, the first collecting portion 31 may collect fibrous materials having a relatively short length, and the second collecting portion 32 may collect fibrous materials having a relatively long length. In another embodiment, both the first collecting portion 31 and the second collecting portion 32 may collect fibrous materials having a relatively long length.

The field generator 4 generates a field in a direction from the introduction unit 2 toward the collecting unit 3. For example, the field generator 4 generates an electric field. When the fibrous materials are DNA and have a negative (−) polarity, the field generator 4 may apply a bias such that the introduction unit 2 has a negative (−) polarity and the collecting unit 3 has a positive (+) polarity. By this electric field, the fibrous materials such as DNA can move towards the collecting unit 3.

When the laser device 5 irradiates laser light to the moving path providing section 1, the capture zone-providing segment 11 is provided with a capture zone caused by a light field due to a nanoplasmon phenomenon. As a result, it can be easily understood that fields of light (light fields) are provided in a lattice structure, and that the lattice structure is the same as in the capture zone-providing segments 11. As described above, even a milliwatt-level laser device can create capture zones for biological particles using a nanoplasmon phenomenon.

Operation of the apparatus for separating fibrous materials according to this embodiment will be described.

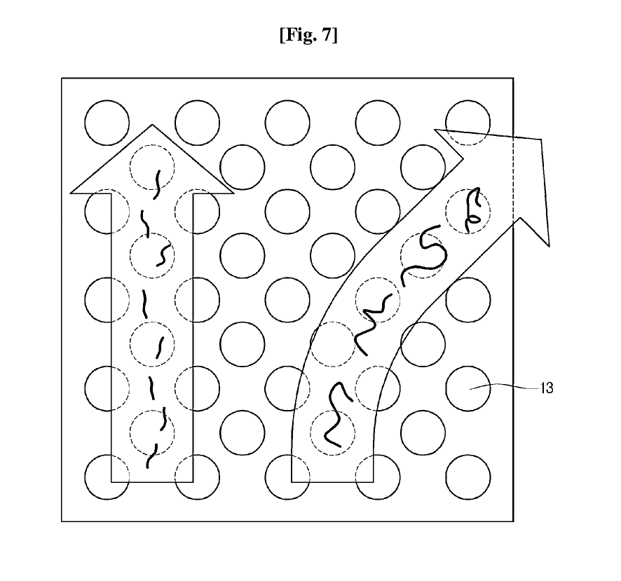

When the laser device 5 irradiates laser light to the moving path providing section 1, capture zones are created in the capture zone-providing segments 11, which are provided as holes, by a light field (see 13 of FIG. 7). The capture zones caused by the light field can capture the fibrous materials.

When the fibrous materials such as DNA are introduced into the moving path providing section 1 through the introduction unit 2, the fibrous materials are subjected to force due to an electric field created by the field generator 4, force due to Brownian motion, and force due to the light field. Here, the force due to the light field causes the fibrous materials to be trapped in any one of the capture zones created by the light field, and the force due to Brownian motion and the force due to the electric field force the fibrous material to move.

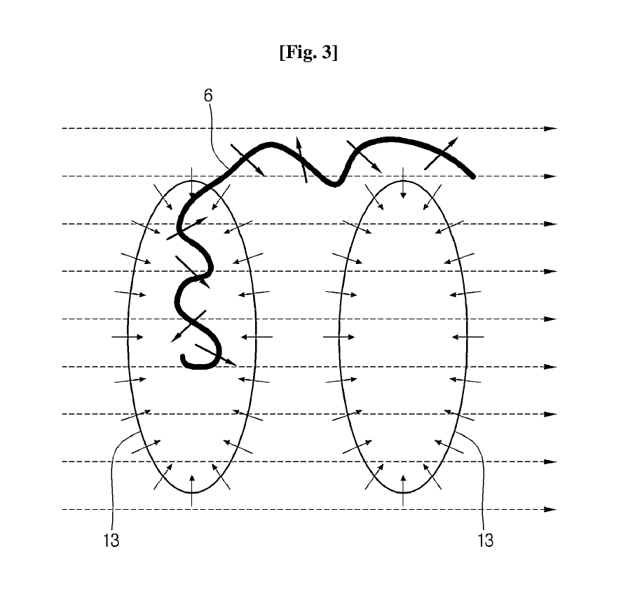

FIGS. 3 to 6 show a movement process of fibrous materials in a pair of adjacent capture zones.

In FIGS. 3 to 6, dashed arrows indicate force due to the electric field, thin solid arrows indicate the force due to the light field in the capture zone, and thick solid arrows indicate the force due to Brownian motion. The light field can be understood as the shape of the capture zone when viewed from a side thereof. If a fibrous material is at least partially included in a capture zone due to the light field, it can be determined that the fibrous material is captured.

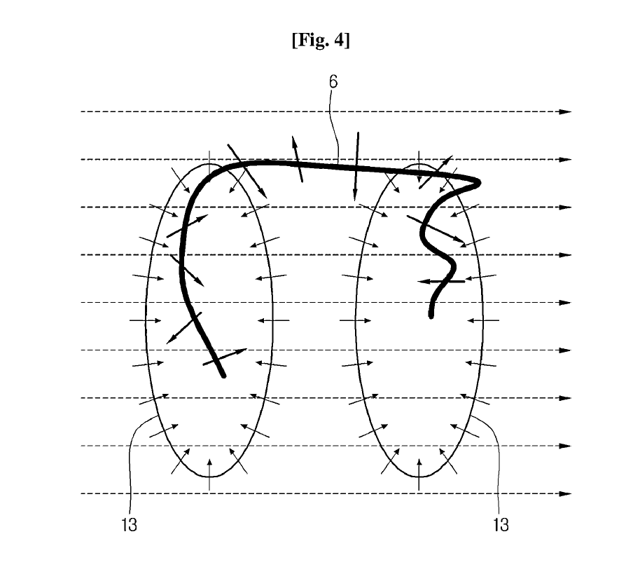

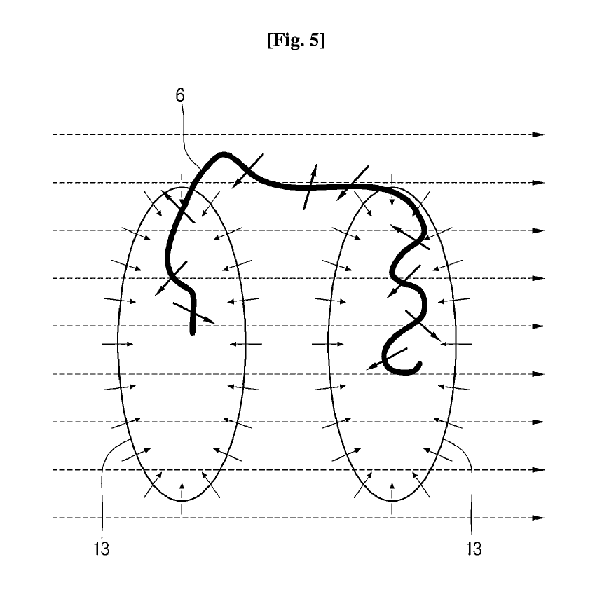

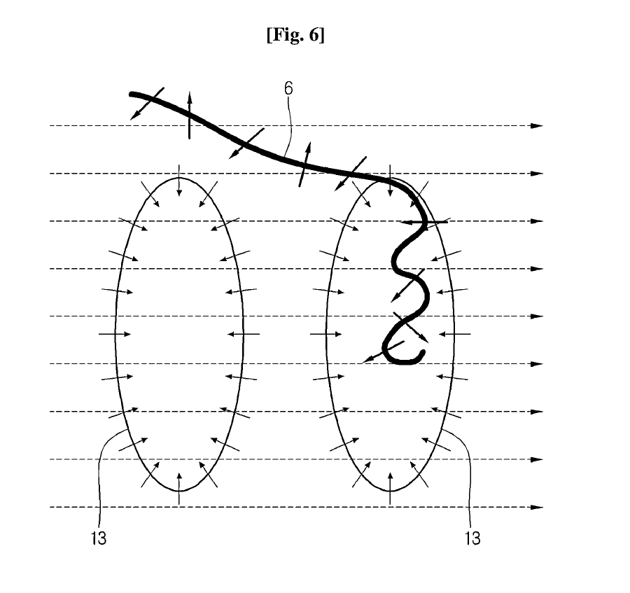

Referring to FIG. 3, when the fibrous material is completely included in the capture zone, the fibrous material is placed in the capture zone even when receiving the force due to Brownian motion and the force due to the electric field. However, when the fibrous material is partially exposed outside of a boundary line of the capture zone due to a long length of the fibrous material, the exposed portion performs relatively large Brownian motion. Referring to FIG. 4, a state in which an exposed end of the fibrous material performing free motion is captured in a right capture zone adjacent thereto is illustrated. Referring to FIG. 5, it can be seen that a left end of the fibrous material partially departs from a left capture zone due to Brownian motion, and most of a chain moves into the right capture zone. Referring to FIG. 6, it can be seen that, as time elapses, the left portion of the fibrous material completely departs from the left capture zone. In conclusion, the fibrous material moves from the left capture zone into the right capture zone. This movement can be viewed as a form of jumping between zones. Force moving the fibrous material may include the force due to Brownian motion and the force due to the electric field, and force allowing the fibrous material to stay in the certain capture zone may include the force due to the light field.

Here, a design factor determining the occurrence of jumping may be the distance between two capture zones. For example, a distance (L1) between the capture zone-providing segments 11 in the vertical direction may be 200 nm, a distance (L2) between the capture zone-providing segments 11 in the diagonal direction is 140 nm, and the fibrous materials are different kinds of fibrous materials having a length of 16 nm and a length of 160 nm, respectively. Here, the fibrous material having a short length of 16 nm may be captured in any one of the capture zone-providing segments. However, the fibrous material having a long length of 160 nm moves from any one of the capture zone-providing segments to another adjacent capture zone-providing segment. Of course, the short fibrous material also does not permanently stay in any one of the capture zone-providing segments and departs therefrom by force due to Brownian motion, and the departing fibrous material is moved by force due to an electric field. Depending on the lengths of the fibrous materials, two flow directions are generated. First, in the case that the length of the fibrous material is shorter than that of the capture zone-providing segment so that the fibrous material is completely included and captured in any one of the capture zone-providing segments, when the fibrous material departs from the capture zone-providing segment by the force due to Brownian motion, the fibrous material flows upwards by the force due to the electric field in FIG. 2. On the contrary, when the length of the fibrous material is longer than that of the capture zone-providing segment so that the fibrous material is not completely included in any one of the capture zone-providing segments, a portion of the fibrous material, which is not captured, wanders due to Brownian motion and moves into an adjacent capture zone-providing segment having a relatively close distance in a diagonal direction from the one capture zone-providing segment. In addition, once the portion of the fibrous material begins to move in a certain diagonal direction, there is a strong possibility that the fibrous material continuously moves in the diagonal direction.

As a result, the long fibrous material captured in any one of the capture zone-providing segments moves into the adjacent capture zone-providing segment 11 close to the one capture zone-providing segment (for example, 140 nm) in the diagonal direction instead of the adjacent capture zone-providing segment 11 far from the one capture zone-providing segment (for example, 200 nm) in the vertical direction. This process continues until the fibrous material passes through the whole area of the moving path providing section 1.

FIG. 7 is a diagram showing two types of fibrous materials passing through a moving path providing section.

Referring to FIG. 7, it can be seen that a short fibrous material continues to move in the vertical direction, and a long fibrous material moves in the diagonal direction. Although FIG. 7 illustrates that the fibrous material moves only in a rightward diagonal direction, it should be understood that the fibrous material may also move in a leftward diagonal direction. However, the fibrous material moving in the leftward diagonal direction does not reach the collecting portion 32 and flows out of the moving path providing section 1, and thus does not affect purity of the fibrous material.

Once the long fibrous material begins to move in a certain diagonal direction, there is a strong possibility that the fibrous material continuously moves in the diagonal direction due to inertia. Thus, the long fibrous material may continue to move in the certain diagonal direction. In addition, if necessary, when the long fibrous material is not smoothly moved in the certain diagonal direction (for example, in the rightward diagonal direction), an additional weak field may be applied in the right direction, or the direction of the applied electric field may be slightly changed in the right direction. In these cases, although straight movement of the short fibrous material in the vertical direction can be obstructed, it is clear that, since an infinitely large number of capture zone-providing segments 11 are formed in the moving path providing section 1, locations at which averagely long and short fibrous materials are separated can be identified from each other

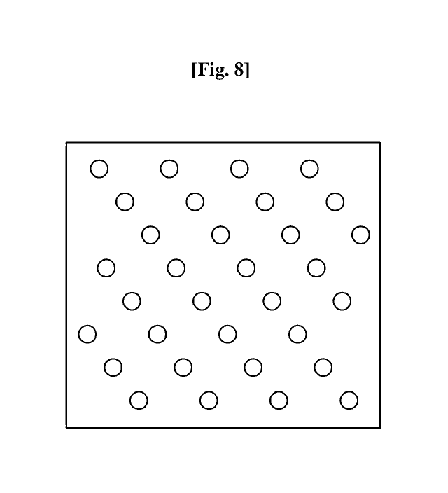

FIG. 8 is a plan view of a moving path providing section according to another embodiment of the present invention.

Referring to FIG. 8, lattice arrangement of trapping sites can be changed into a 45 degrees dotted line arrangement that is repeated periodically. In this way, all fibrous materials longer than the adjacent trapping sites will move along the dotted line direction and the fibrous materials that are shorter than the adjacent trapping sites will move in the direction of the field.

Depending on the above operation, in FIG. 1, the short fibrous materials may be obtained in the first collecting portion 31, and the long fibrous materials may be continuously and purely obtained in the second collecting portion 32. In another embodiment, the long fibrous materials may be continuously and purely obtained in the first and second collecting portion 31 and 32. The short fibrous materials are not collected and they can flow in the external field direction.

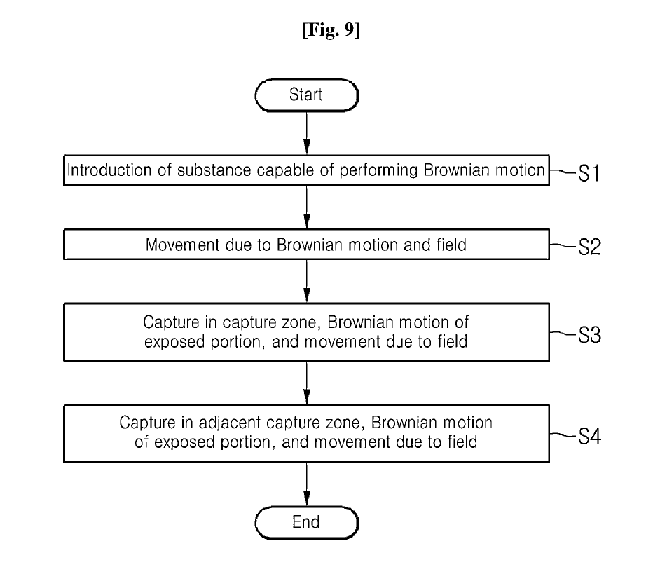

FIG. 9 is a flowchart of a method for separating fibrous materials.

Referring to FIG. 9, a fibrous material capable of performing Brownian motion is introduced into a moving path providing section (S1). The fibrous material introduced into the moving path providing section is moved by force due to Brownian motion and due to a field (S2). Then, after the fibrous material is moved a certain distance, the fibrous material is captured in a capture zone-providing segment while the force due to Brownian motion and the field is continuously applied thereto (S3). The fibrous material is moved into an adjacent capture zone-providing segment by the force due to Brownian motion and the field, and a moving path of the fibrous material varies depending on the length of the fibrous material (S4). Even in this state, the force due to Brownian motion and the field is continuously applied to the fibrous material. Such movement is continuously performed until the fibrous material reaches a collecting unit.

The present invention may be embodied in other ways. Hereinafter, other embodiments of the invention will be described in detail without departing from the spirit and scope of the invention.

First, an electric field generated by the field generator may be substituted with another field. For example, the electric field may be substituted with a magnetic field and, in this case, the fibrous material may be covered with iron. Alternatively, the field generator may be removed, and a fluid field may be applied by allowing fluid to flow from the introduction unit toward the collecting unit at a constant flow rate. Alternatively, the field generator may be removed, and a gravity field may be applied to the fibrous material to force the fibrous material to be moved toward the collecting unit by gravity.

Next, the method in which the capture zones are provided by the light field may be substituted with other methods. For example, the laser device may be removed, and the capture zone-providing segments may be prepared as holes, such that a fluid can flow through the holes. In this case, since a fluid field is generated by the fluid, the fibrous material may be captured. However, in this case, the flow rate of the fluid is precisely controlled so as not to be excessively rapid. Alternatively, a DNA binding protein may be provided to the capture zone-providing segment such that DNA can be temporarily bound thereto.

Next, examples of the fibrous material may include other fibrous materials, such as carbon nanotubes, general proteins, and the like, as well as DNA. Here, a polar material may be attached to polarity-free materials such that the polarity-free materials have polarity. Fields that do not exhibit polarity (for example, a gravitational field) do not need a polar material to be attached thereto.

In addition, to improve separation effects, vibration may be applied to the moving path providing section.

Further, by adjusting the direction of applying the field, the distances between the capture zones may be adjusted in different ways in the diagonal direction and in the vertical direction.

According to embodiments of the invention, the apparatus and method for separating fibrous materials may have improved safety and improved purity of separated fibrous materials, and enables separation of large amounts of fibrous materials through a simple separation process. In addition, the present invention provides various other advantages that can be inferred from the spirit of the invention. Therefore, the apparatus and method according to the embodiments of the invention may significantly increase yield.

Although some embodiments have been described herein, it will be apparent to those skilled in the art that these embodiments are given by way of illustration only, and that various modifications, changes, alterations, and equivalent embodiments can be devised without departing from the spirit and scope of the invention. The scope of the invention should be limited only by the accompanying claims and equivalents thereof.

What is claimed is:

1. An apparatus for separating fibrous materials comprising:
an introduction unit through which fibrous materials capable of performing Brownian motion are introduced;
a moving path providing section prepared in a state in which at least one field causing movement of the fibrous materials in a predetermined direction is applied thereto, and providing a moving path for the fibrous materials;
at least three capture zone-providing segments provided to the moving path providing section so as to at least temporarily capture the fibrous materials; and
a collecting unit collecting the fibrous materials having passed through the moving path providing section.

2. The apparatus according to claim 1, wherein the at least one field includes at least one of an electric field, a magnetic field, a gravitational field, and a fluid field.

3. The apparatus according to claim 1, wherein capture zones provided by the at least three capture zone-providing segments are formed by at least one of a light field due to a nanoplasmon phenomenon, a fluid field, and a DNA binding protein.

4. The apparatus according to claim 1, wherein a distance between the at least three capture zone-providing segments close to a direction of applying the at least one field is greater than that far from the applied direction of the at least one field.

5. A method for separating fibrous materials comprising:
moving fibrous materials along a field; and
at least temporarily capturing the fibrous materials in at least three capture zones separated from each other.

6. The method according to claim 5, wherein a distance between the at least three capture zones close to a direction of applying the field is larger than that far from the direction of applying the field.
